(19) United States
(12) Patent Application Publication
(10) Pub. No.: US 2005/0171404 A1
(43) Pub. Date: Aug. 4, 2005

(54) Surgical retractor having suture control features

(76) Inventor: Hans A. Mische, St. Cloud, MN (US)

Correspondence Address: Kagan Binder, PLLC;Maple Island Building, Suite 200, 221 Main Street North, Stillwater, MN, 55082, US

(21) Appl. No.: 11/031,055

(22) Filed: Jan. 7, 2005

Related U.S. Application Data
(60) Provisional application No. 60/535,011, filed on Jan. 8, 2004.

(51) Int. Cl. A61B 1/32

(52) U.S. Cl. 600/231

(57) ABSTRACT

A surgical retractor system comprising an annular frame having an inner edge and an outer edge having a plurality of spaced notches having a first width, wherein the inner edge defines an inner area of the frame, and at least one suture control device positioned within at least one of the notches for holding at least one piece of suture material, wherein the suture control device has an opening with a second width that is smaller than the first width. The suture control device may comprise an insert having an outer surface adjacent to an inner surface of the at least one notch in which it is positioned. Also, the annular frame may have a planar surface at the plurality of spaced notches, wherein the at least one suture control device is positioned so that it does not extend above the planar surface of the annular frame.

### REFERENCE TO RELATED APPLICATIONS

This application claims the benefit of U.S. Provisional application having Ser. No. 60/535,011, filed Jan. 8, 2004, entitled “SURGICAL RETRACTORS”, which application is incorporated herein by reference in its entirety.

### TECHNICAL FIELD

The present invention relates to surgical instruments and more specifically to surgical retractors for use in various surgical procedures that require access to internal body organs or tissues by a surgeon.

### BACKGROUND OF THE INVENTION

During many surgical procedures, an incision is made through the skin and into the tissue of the patient in order to provide access to internal organs. The surgical incision is then separated and retracted in some way to maintain the opening in an open or exposed condition to thereby provide access to the operating site by a surgeon. In some cases, as the surgeon cuts into the tissue, the operating room staff will hold the tissue away from the operating field using retractors. One way this is accomplished is with the use of one-piece metallic retractors that typically retract the wound in a non-yielding manner that can result in tearing and bruising of the tissue. Another way retraction is accomplished is through the use of “stay” sutures that are placed through the tissue. These sutures are typically controlled by clamping a device such as a hemostat to the end of each of the sutures so that the hemostats or other devices can be pulled in the desired directions to widen the incision via the movement of the sutures. The sutures then can also maintain the tissue in an open condition. The site is then more readily accessible by the surgeon for reaching the affected organs, tissues, muscles, and the like, for performing the necessary surgery and/or for the implantation of various devices into the body. However, the use of these sutures and attached devices can clutter the surgical field for the surgeon and can be difficult to maintain in their desired positions due to the variations in the skills of the operating staff, especially in cases where the surgery takes an extended period of time.

Thus, various types of retractors that require less constant interaction by the operating staff are available for different types of surgeries, where the devices are designed to accommodate the particular area of the body on which surgery will be performed. These retractors are typically designed to not obstruct either visual or physical access by the surgeon. For example, retractors used in spinal surgery require a retractor that is strong enough to overcome a relatively large muscle mass that needs to be dissected away from the field of exposure, such as a retractor that includes large blades or paddles that move the muscles and tissues away from the spine to provide the necessary access by the surgeon.

In other cases, a retractor frame may be used in an area that is designed to conform to the portion of the body is provided, which may be used with multiple retractor devices or stays. One particular type of frame that may be used for penile-scrotal surgeries that require dissection and exposure of the corpora includes a retractor frame that is particularly designed for the genital area of the body. The retractor frame can be placed against the skin of the patient around the surgical site either before or after the incision is made by the surgeon. Elongated retractor stays, which are typically made of an elastic material, can then be positioned so that one end of the stay engages with the tissue at the incision and the other end is attachable to the retractor frame. These retractor stays may be repositioned throughout the surgery, as desired, to provide adequate access to the surgical site for the surgeon. In one particular retractor frame design, the frame is provided with a plurality of notches spaced about the periphery of the frame, while the stays include a tissue-holding device (e.g., a hook portion) at one end of an elastic member. The surgeon can position the tissue-holding device within the incision, then use the elastic member to adjust the traction applied to the tissue by the placement of the elastic member within the notches. Retractor frame designs known in the art include those having rigid, one-piece constructions that are contoured generally to fit a particular area of the human body, and those frames that include portions that are moveable relative to one another (e.g., two portions that pivot about two pivot points). Other retractor frames are more capable of being adjusted or reconfigured to match the contours of the surgical site, such as with constructions having two or more pieces with malleable portions connecting them to each other.

While elastic members or stays can often provide the necessary traction for maintaining an incision in an open condition, there is a need to provide additional ways of using sutures in a controlled manner to additionally or alternatively keep an incision in an open condition. Such suture control can provide surgeons with additional options for performing surgery that uses surgical materials that are readily available in the operating room.

### SUMMARY OF THE INVENTION

In one aspect of this invention a surgical retractor system comprising an annular frame having an inner edge and an outer edge having a plurality of spaced notches having a first width, wherein the inner edge defines an inner area of the frame, and at least one suture control device positioned within at least one of the notches for holding at least one piece of suture material, wherein the suture control device has an opening with a second width that is smaller than the first width. The suture control device may comprise an insert having an outer surface adjacent to an inner surface of the at least one notch in which it is positioned. Also, the annular frame may have a planar surface at the plurality of spaced notches, wherein the at least one suture control device is positioned so that it does not extend above the planar surface of the annular frame.

In another aspect of the invention, a surgical retractor system is provided, comprising an annular frame having an inner edge, an outer edge having a plurality of spaced notches, and first and second opposite surfaces, wherein the inner edge defines an inner area of the frame, and at least one suture control device extending from at least one of the first and second opposite surfaces for capturing at least one suture. The at least one suture control device may comprise a raised knob portion that may include a notch or channel. The at least one suture control device may comprise a coiled member.

### BRIEF DESCRIPTION OF THE DRAWINGS

The present invention will be further explained with reference to the appended Figures, wherein like structure is referred to by like numerals throughout the several views, and wherein:

FIG. 1 is a top plan view of a surgical frame positioned over a surgical incision that is held open with the use of a plurality of sutures held in place by suture control features, in accordance with the present invention;

FIG. 2 is top plan view of a portion of a surgical frame including one embodiment of suture control features of the present invention;

FIG. 3 is top plan view of a portion of a surgical frame including an auxiliary knob used for suture control;

FIG. 4 is a side view of one embodiment of a suture control device extending from a surgical frame, where the suture control device includes horizontal slots for receiving sutures;

FIG. 5 is a side view of one embodiment of a suture control device extending from a surgical frame, where the suture control device includes at least one vertical slot for receiving sutures;

FIG. 6 is a top plan view of a portion of a surgical frame including an additional narrow notch for use as a suture control device; and

FIG. 7 is a top plan view of a portion of a surgical frame including a coiled device for use in suture control.

### DETAILED DESCRIPTION OF THE PREFERRED EMBODIMENTS

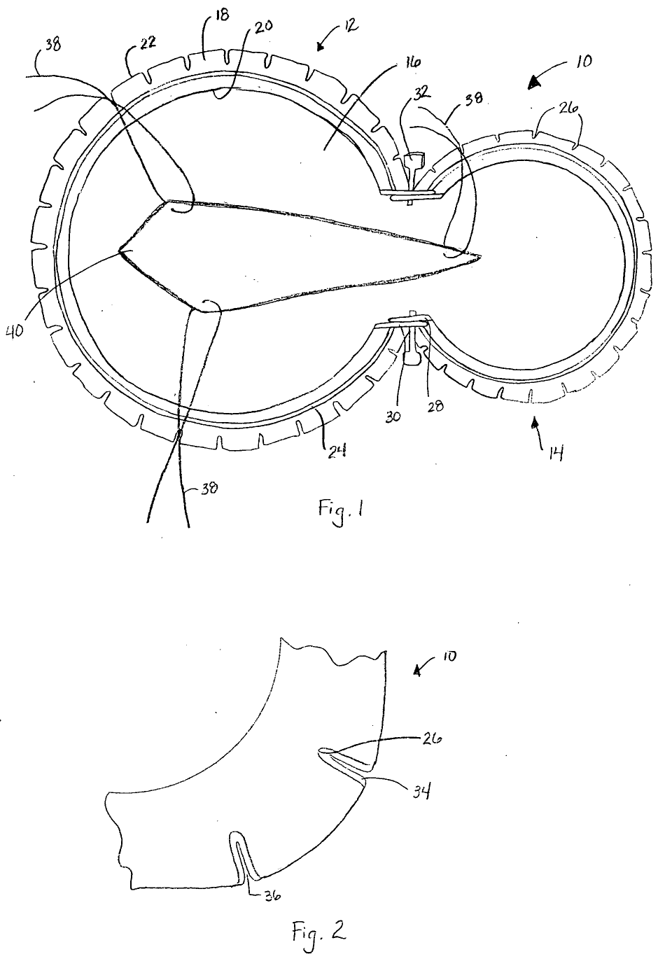

Referring now to the Figures, wherein the components are labeled with like numerals throughout the several Figures, and initially to FIGS. 1 and 2, one preferred configuration of a surgical frame 10 is shown. Surgical frame 10 generally includes a first portion 12 that is generally a truncated portion of a circular or arcuate shape, and a second portion 14 that is somewhat smaller in diameter than first portion 12, but is also generally a truncated portion of a circular or arcuate shape. The two portions 12, 14 are positioned relative to each other to make a generally annular frame 10 having an inner area 16 in which the surgical procedure will be performed, as explained in further detail below. The peripheral shape of the frame 10 is generally a “figure 8”, as shown; however, it is understood that the peripheral shape of the two portions 12, 14 can vary considerably in shape and size from that shown to form a different outer peripheral shape of the frame 10. For example, the two portions 12, 14 may be the same size as each other, one or both portions 12, 14 may be more oblong or oval in shape, one or both portions 12, 14 may be more square or rectangular in shape, one or both portions may be more V-shaped, or the like. In any case, the size and shape of the first and second portions 12, 14 should be chosen to accommodate the general contours of the portion of the body on which it will be positioned.

The surgical frame 10 has an upper surface 18 and an opposite lower surface (not shown). The upper surface 18 is substantially planar and extends from an inner edge 20 to an outer edge 22. The upper surface 18 may be substantially flat, but preferably angles somewhat upwardly from the inner edge 20 to the relatively higher outer edge 22. Alternatively, the upper surface 18 may be curved generally upwardly from the inner edge 20 to the outer edge 22, or the surface 18 may extend at one angle or curve from the inner edge 20 to an inner ridge 24, then extend at another angle or curve from the inner ridge 24 to the outer edge 22. The surface 18 may alternatively have different contours or angles provided to conform generally to the contours of the portion of the body on which the frame 10 will be used.

The second frame portion 14 preferably further includes extension portions 28 extending from each end of the truncated circular frame portion 14, which preferably include a relatively flat piece that extends in a generally perpendicular direction from the plane of the frame portion 14. Similarly, the first frame portion 12 includes extension portions 30 extending from each end of the truncated circular frame portion 12, which also preferably include a relatively flat piece that extends in a generally perpendicular direction from the plane of the frame portion 12. Each of the extension portions 28, 30 preferably include a connection hole through their thicknesses (not shown) that may optionally be threaded, or may be a simple through-hole. As shown, the extension portions 28 of the frame overlap the extension portions 30 so that their connection holes are aligned for accepting a connection member 32 on each side of the frame. The connection members 32 may be threaded to mate with threads inside the connection holes so that the connection members 32 can be loosened to allow relative movement between the first frame portion 12 and the second frame portion 14, then can be tightened once the frame portions 12, 14 are in their desired positions relative to each other by rotating the wing portion of the connection members 32 to fix the extensions 28, 30 in a final position and lock the frame in position. A variety of other configurations for locking the first and second frame portions 12, 14 relative to each other are contemplated, such as using an malleable connector between the pieces, using connection members that are threaded only at one end to accept a nut that can be tightened to lock the frame portions 12, 14 relative to each other, and the like.

The surgical frames may be reusable and made of a material that can be sterilized at the point of use, such as aluminum, stainless steel, titanium, or other medical-grade metals. Alternatively, the frames may instead be disposable and therefore made of a material that is not designed to be sterilizable at the surgical site, such as a plastic material that is sufficiently strong to support the use of any types of stays that are connected thereto without causing bending or buckling of the frame. One example of such a material from which a disposable frame may be made is a plastic or resin material, and one preferred example of which is a polyphenylene ether/polystyrene blend (PPE/PS) commercially available under the trade name “Noryl” and available from GE Plastics of Pittsfield, Mass. These disposable retractor frames can be pre-sterilized and packaged singly in pouches for delivery to the sterile surgical field, and are typically weigh less than similarly shaped and sized reusable frames, but should still be made of a material that provides adequate structural rigidity for the surgeon.

The outer edge 22 of the frame 10 includes a plurality of notches 26 that extend through the frame 10 and are spaced around its periphery. As shown, the notches 26 are evenly spaced from each other; however, the notches 26 may instead be spaced at different intervals from each other around the periphery of the surgical frame 10. The notches 26 can be designed to accommodate the efficient organization of sutures and elastic stay hooks, when such sutures and hooks are desired for a particular surgical procedure. Examples of stay hooks that can be used with the surgical frames of the type used in the present invention include elastic stay hooks that are commercially available from Lone Star Medical Products, Inc. of Stafford, Tex., which generally include an elongated elastic band having a hook or multiple hooks at one end for engagement with the tissue surrounding the incision. The hooks can be small or large, may include single or double hooks, may be sharp or blunt, may include solid blades, and/or may include blunt rakes with two or more fingers, as desired for the particular surgery. The material used for the sutures can be any known thread-like, medical grade material used for stitching or closing wounds, or may be another elongated thread-like material that can be threaded through the skin and tissue for holding an incision open in accordance with the suture control methods and devices of the present invention.

FIG. 1 further illustrates the frame 10 positioned to surround an open incision 40 that was either cut by a surgeon or that occurred due to an injury or accident. Multiple sutures 38 are inserted through the skin and some thickness of the underlying tissue around the incision 40 to provide specific points in the skin and tissue that can be pulled in a direction to move parts of the skin and tissue away from other parts of the skin and tissue on opposing sides of the incision 40. The sutures 38 are threaded completely through a desired thickness of skin and tissue so that two ends of the suture 38 are available to be grasped by the surgeon. The sutures should preferably not be pulled toward the center of the incision because this may cause the skin and tissue to rip or tear. Once the sutures 38 are inserted through the skin and tissue, the surgeon can then pull each of the sutures 38 to the desired tension to sufficiently open the incision. The ends of each of the sutures 38 can then be inserted into the notches 26 and held in place with a suture control device, several embodiments of which are described below. The sutures 38 can preferably then be maintained in their desired position without additional manipulation by the surgeon and/or surgical staff. However, the sutures 38 may optionally be further adjusted or repositioned in order to provide more or less tension on the edges of the incision throughout the surgical procedure. FIG. 1 illustrates three separate sutures 38; however, it is understood that more or less than three sutures may be used in a surgical procedure.

With continued reference to FIG. 1, at least one of the notches 26 may include a suture control device, and preferably a significant number of the notches will contain such a device to provide the surgeon with a choice of multiple locations for securing sutures. The surgical frame may have some notches with suture control devices and others that are designed to accept elastic stays or other devices. For one example, notches having a suture control device can alternate with notches that do not have suture control devices around the perimeter of the first and second frame portions 12, 14, in order to provide the surgeon with a sufficient number of locations for securing both elastic stays or sutures, as desired. A wide variety of the arrangement of notches having suture devices is possible and can be selected or designed to best accommodate the surgical procedure that will be performed. Further, a single surgical frame may be provided with the same or different types of suture control devices around its perimeter.

In accordance with the embodiment of FIG. 1, at least some of the notches 26 of the surgical frame 10 will include a suture control device within the open space or gap of the notch 26. One preferred configuration of a control device 34 is better illustrated in the enlarged portion of the surgical frame 10 shown in FIG. 2. As shown, the control devices 34 consist of an insert having a small slit or gap 36 that has a width that is smaller than the width of the notch 26 in which it is inserted. The gap 36 is preferably sufficiently wide and deep to accommodate at least one piece of suture material, and preferably is wide and deep enough to accommodate two pieces of suture material (e.g., the two end portions of a length of suture material). In accordance with the present invention, however, the gap 36 is preferably also small or narrow enough that sutures positioned therein are securely held within the gap 36 until the surgeon desires to remove the suture material from the gap 36. That is, once the sutures are inserted into the gap 36, the sutures preferably should not move or slip substantially until a positive action of the surgeon or other user disengages the sutures from the gap 36.

One embodiment of the suture control device 34 is constructed from material such as a silicone polymer, for example, that is cut or slit a sufficient distance to provide the gap 36 in a direction that coincides generally with the center axis of the notch 26 in which it will be inserted. This gap 36 may be formed as merely a slit in the device 34, in which case it is advantageous for the material from which the device is made to be relatively flexible so that it will move or flex when suture material is pressed into the gap 36. In other words, the two sides of the gap 36 may actually be touching each other when no suture material is inserted therein, then the sides of the gap 36 can be moved away from each other to accommodate the thickness of the suture material inserted therein. Alternatively, an open space may be provided between the two sides of the gap 36 whether or not suture material is inserted into the gap 36 such that the two sides of the gap are never in contact with each other. Further, the gap 36 may either have a constant width along its length, or it may be tapered or otherwise differing in shape along its length. In any case, the gap 36 may be preformed in the suture control device 34 before it is positioned within the notch 26, or the operation that forms the gap 36 may be performed after the suture control device 34 is positioned within the notch 26.

The suture control device 34 can be a preformed insert that slips into the notch 26 and secured therein with an adhesive or other fastening means, whether the gap 36 is formed therein or if the gap 36 is subsequently created. Alternatively, the material from which the device 34 is constructed may have sufficient adhesive properties to allow it to adhere to the interior portion of the notch 26 without any additional adhesive materials. In any case, the suture control device 34 preferably is made of a material that allows the surfaces of the gap 36 to hold or “grab” the suture material when it is inserted into the gap 36. Materials other than the exemplary silicone polymer may be used for the suture control device 34, such as rubber-like materials, or the like.

The surfaces inside the gap 36 of the suture control device 34 are preferably generally smooth to allow the sutures to be easily inserted by the surgeon without snagging or tearing. However, the surfaces of the gap 36 can preferably also provide at least some frictional properties that can help to keep the suture from slipping out of the gap 36. These frictional properties may be provided by the material from which the control device 34 is made (e.g., a material that is relatively “sticky”). Alternatively, the surfaces of the gap 36 can be textured at least slightly to help prevent sutures from sliding out of the gap 36. In any case, the surfaces of the gap 36 should not have sharp edges or protrusions that would tend to tear or break the suture material.

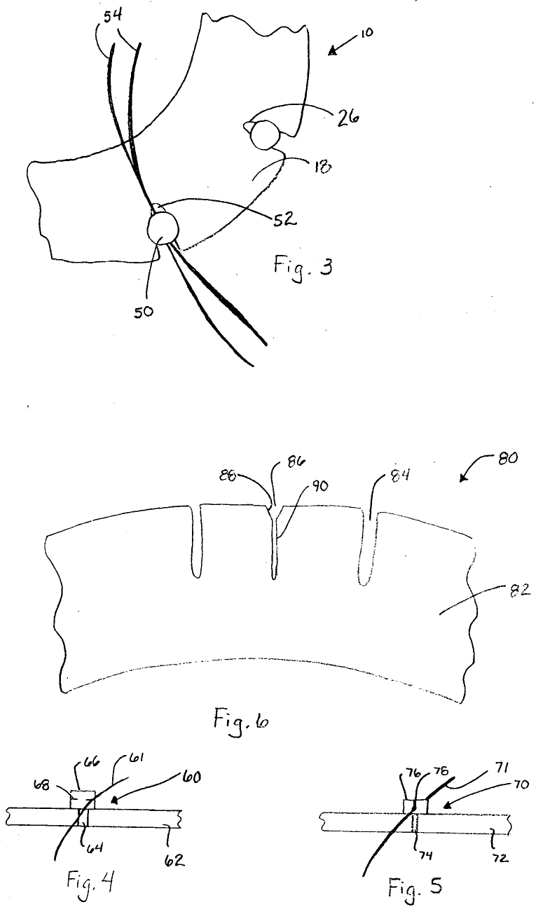

Another preferred embodiment of a suture control device 50 is illustrated in the enlarged portion of the surgical frame 10 shown in FIG. 3. As shown, the suture control device 50 consists of a knob or protrusion that extends from the internal area of each notch 26. The control device 50 may be a bead of silicone material, for example, that is attached to the top surface 18 of the frame. Alternatively, the control device 50 may be a preformed piece of plastic, metal, or some other material that is adhered or otherwise attached to the top surface of the frame, the inside of the notch 26, or both the top surface 18 of the frame and the inside of the notch 26, as desired. As shown, a slight space 52 may be provided between the notch 26 and the control device 50 by the positioning of the control device 50. When such a space 52 is provided, it is preferably large enough to accept the suture pieces 54 that are to be secured within the notch 26 so that the suture pieces 54 can essentially be threaded through the space 52, if desired. After the sutures 54 are threaded through the space 52, they can then be pulled into a space between the control device 50 and the top surface 18 of the frame 10 to secure the sutures in place. It is also contemplated that no space is provided between the control device 50 and the top surface 18 of the frame 10 so that the sutures 54 would instead need to be secured within the notch 26 in another manner, such as by wrapping the sutures around the control device 50, threading the sutures through a hole or other access passage in the control device itself, and the like. Alternatively, the control device 50 can be spaced slightly from the top surface 18 of the surgical frame 10 by a sufficient distance that the sutures can be slid under the knob and secured between the bottom of the control device 50 and the top surface 18 of the frame 10 without threading the sutures 54 through the space 52. In a case such as this where the sutures 54 do not need to be threaded through a space in the notch 26, such a space 52 need not be provided and the control device may instead be inserted completely in the notch 26 to eliminate any space 52.

In order to maintain the sutures in their desired positions relative to the frame and its notches, the control device 50 may be configured in a number of different ways. One example, which is shown in FIG. 3, includes a control device 50 that is a generally circular disk that is secured within the notch 26 in a manner that is either adjustable or permanent. A specific variation of the control device 50 is illustrated as control device 60 in FIG. 4. Control device 60 consists of a knob or extension 66 that extends from one surface of a surgical frame portion 62. As shown in this embodiment, the knob 66 extends generally from a notch 64 in the frame 62; however, the knob 66 may instead be attached to the frame 62 at some point that is not associated with a notch. Knob 66 includes at least one horizontal suture notch 68 that is preferably sized to be wide enough to accept one and preferably two or more pieces of suture material. In this figure, one suture 61 is illustrated as positioned within the suture notch 68 on one side of the knob 66, but it is possible that more than one suture 61 be inserted into this suture notch 68 and/or that other sutures can be inserted in the suture notch 68 on the other side of the knob 66, for example. The suture notch 68 may optionally include a widened area with which the suture can engage to better secure the suture. This widened area may be provided, for example, with a notch 68 that is more of a slit in the knob 66 than an actual gap, and is therefore opened only by the force of a suture being inserted therein. The widened area would then provide a sort of relief area for the suture material that would thereby allow the suture notch 68 to close after the insertion of the suture.

The suture notches 68 in a particular knob 66 may be more of a channel that extends around all or part of the knob 66, if desired, rather than the individual suture notches shown. In either case, the suture material 61 may either be simply inserted one time through a suture notch 68, or may instead be wrapped around the knob 66 more than one time, with the suture optionally being inserted into the notch 68 with each wrap of the suture.

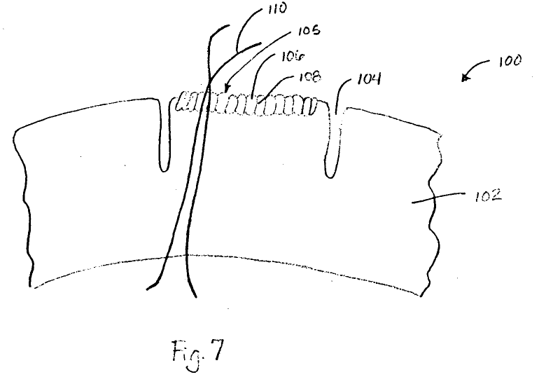

Another variation of the control device is illustrated as suture control device 70 in FIG. 5. Control device 60 consists of a knob or extension 76 that extends from one surface of a surgical frame portion 72. As shown in this embodiment, the knob 76 extends generally from a notch 74 in the frame 74; however, the knob 76 may instead be attached to the frame 72 at some point that is not associated with a notch. Knob 76 includes at least one vertical suture notch 78 that is sized to be wide enough to accept one and preferably two or more pieces of suture material. In this figure, one suture 71 is illustrated as positioned within the suture notch 78, but it is possible that more than one suture 71 be inserted into this suture notch 78. The suture notch 78 may optionally include a widened area with which the suture can engage to better secure the suture, such as at the bottom of the notch 78. This widened area may be provided, for example, with a notch 78 that is more of a slit in the knob 76 than an actual gap, and is therefore opened only by the force of a suture being inserted therein. The widened area would then provide a sort of relief area for the suture material that would thereby allow the suture notch 78 to close after the insertion of the suture. Alternatively, the suture notch may be oriented at an angle that is not vertical as shown in FIG. 5 or horizontal as shown in FIG. 4.

While the above descriptions of FIGS. 3, 4, and 5 refer mainly to the use of various versions of an extending knob for a suture control device, it is understood that other device configurations for securing the sutures are considered within the scope of the present invention. Such alternative device configurations include snaps, pegs, stanchions, loops, miniature twisters similar to those used in marine applications, and the like. Any of these devices may be secured within a notch or may attach or extend from the surgical frame in an area spaced from the notches. It is preferable that any of these suture control devices are designed to be as small as possible while being able to perform their desired functions, in order to not interfere with the surgical process. In addition, the suture control devices may be incorporated into the surgical frame during the molding or other forming of the surgical frame, or may instead be attached in a secondary process such as insert molding or manufacturing. Further, it is preferable that any of these extending suture control devices have surfaces that will not unintentionally damage any suture material that comes in contact with it.

FIG. 6 illustrates an alternative embodiment of suture control in accordance with the present invention in a surgical frame system 80. System 80 includes a surgical frame 82 having a plurality of spaced notches 84, as described above for use with elastic retractor stays. In this embodiment, a suture control device is provided as a suture notch 86, which is an additional notch in the frame specifically designed for securely holding sutures. The suture notch 86 is preferably narrower than the notches 84 that are typically provided for the retractor stays. However, this is not necessarily the case because the notches 84 may be relatively narrow for accepting smaller elastic retractor stays or other devices, and/or the suture notch 86 may be relatively wide so that the notches 84 and suture notches 86 may be the same size or the suture notches 86 may actually be wider than the notches 84. Similarly, the depth of the suture notches 86 and the notches 84 may be the same or different. The notches 86 are preferably provided with an elongated narrow portion 90 at its base, and a wider portion 88 at the edge of the surgical frame 82. In this embodiment, the portion 88 is a v-shaped notch, however, the wider portion 88 may be shaped differently, such as curved, squared, or the like. In any case, the portion 88 is provided as an aid to help the surgeon guide the suture into the narrow portion 90, which can thereby make the insertion of a suture quicker and easier for the surgeon. Thus, the portion 88 may be designed to be only slightly wider than the narrow portion 90, or may the portion 88 be considerably wider than the narrow portion 90, as desired. The forming of the suture notch 86 in the frame 82 may be performed during the molding or forming of the surgical frame 82, or may instead be formed during a secondary operation such as machining, cutting, slitting, or notching with an appropriate piece of machining equipment.

FIG. 7 illustrates an alternative embodiment of suture control in accordance with the present invention in a surgical frame system 100. System 100 includes a surgical frame 102 having a plurality of spaced notches 104, as described above for use with elastic retractor stays. In this embodiment, a suture control device is provided as a suture control coil 105, which is a coiled material 106, such as metal or plastic. The coil 105 is preferably designed so that each successive loop of the coiled material 106 is relatively close to each adjacent loop of the material 106, or the loops of material may all be touching the adjacent loops. In any case, adjacent coils of material 106 can preferably move relatively easily away from each other in response to a piece of suture material being inserted between them. As shown, two ends of a suture 110 are inserted into spaces 108 between coils 106 of the suture control coil 105. Preferably the coils 106 have sufficient spring force that they will tend to move back toward each other after the insertion of suture material, thereby providing a secure hold of the suture material. The suture control coil 105 may have any number of coils 106, and may be positioned on the edge of the surgical frame 102 between two notches 104, as shown, or may extend along more or less of the edge of the surgical frame 102. However, a suture control coil 105 with a large number of coils 106 will provide the surgeon with more choices of places to secure the sutures than a suture control coil with a small number of coils 106. The forming of the suture control coil 105 on the frame 102 may be performed during the molding or forming of the surgical frame 102, or may instead be formed during a secondary operation and subsequently attached to the frame 102.

When using the frames and support members of the present invention in a surgical process, the surgical frame is positioned so that the sterile-draped incision or surgical area is located within the inner area of the frame for access by the surgeon, as shown, for example, in FIG. 1. Any support members or platforms that are moveable can be positioned in their desired orientation for surgery either before or after positioning the frame around the surgical area. Sutures and/or elastic stays can then preferably be inserted into edges of the tissue at various points around the incision and pulled outwardly to achieve the desired tension on the incision edges. The sutures and/or elastic stays can then be inserted into the notches of the frame to provide the necessary counter-traction to stabilize the surgical frame for the remainder of surgery so that no additional mechanical support is needed. As dissection progresses, the position of the elastic stays can be advanced to hook into deeper layers of fascia. New stay hooks can be added throughout the process, or those already in place can be repositioned to achieve full, balanced retraction from any desired angle.

Several examples of alternative surgical frame configurations that provide an optional support member to provide the surgeon with an additional support surface during surgery are described in the copending U.S. Patent application of the present Assignee filed on even date herewith, having U.S. Ser. No. ______, entitled “SURGICAL RETRACTOR WITH INTERMEDIATE SUPPORT MEMBERS”, Attorney Docket No. AMS0057/US, which is incorporated herein by reference in its entirety. For one example, the frame can be provided with a support member having a slot or opening at each end that slides, snaps, or otherwise attaches to the frame. For another example, the frame can include a curved support member that is generally shaped to fit the anatomy of the patient. This or any other support members also optionally include a plurality of notches along at least one of their edges. These notches provide the surgeon with additional locations to which the sutures, elastic stays, or other surgical components may be attached, if desired. Thus, the support members can provide the dual function of being a supporting platform during the surgical process while also serving as a frame piece to which elastic stays can be attached.

The frames used with the present invention can have a wide variety of arrangements for notches around its periphery, where the notches can be identical in size and shape and spaced evenly around the periphery, or may instead include a wide variety of sized, shaped, and spaced notches around the frame periphery. It may be advantageous to provide a frame system including insertable and replaceable suture control devices from which a surgeon can choose for a particular surgery and patient. That is, any of the versions of the suture control device may be molded or assembled as part of the frame that is provided to a surgeon, but could be instead be insertable and removable devices that can be done on site so surgeon can custom design the device. This type of system would be particularly advantageous for reusable frames so that the surgeon could use custom designed frames with or without suture control devices for multiple unique surgeries. However, systems including a choice of several suture control devices can also be useful for disposable frame systems so that the surgeon can choose the type of suture control device that would best suit a particular surgery at the time of surgery for a frame that is customized to the patient.

The surgical frames shown and described herein are directed generally to surgical techniques and devices that are used for penile-scrotal surgeries however, the retractor frames and features thereof described relative to the present invention can also be used with retractor frames for other surgeries, such as vaginal, urological, colorectal, perineal, hand, foot, plastic reconstructive, vascular, head and neck, and other soft tissue surgeries. In these cases, the outer periphery of the frame is preferably sized and shaped to accommodate the contours of the part of the body with which it will come in contact. In these cases, any support members or strap components can be positioned along the frame periphery in one or more locations that provide the surgical advantages described above, such as providing a platform with sufficient strength and properly positioned for supporting a particular body part.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

1. A surgical retractor system comprising:
an annular frame having an inner edge and an outer edge having a plurality of spaced notches having a first width, wherein the inner edge defines an inner area of the frame; and
at least one suture control device positioned within at least one of the notches for holding at least one piece of suture material, wherein the suture control device has an opening with a second width that is smaller than the first width.

2. The surgical retractor of claim 1, wherein the at least one suture control device comprises an insert having an outer surface adjacent to an inner surface of the at least one notch in which it is positioned.

3. The surgical retractor of claim 1, wherein the annular frame has a planar surface at the plurality of spaced notches, and wherein the at least one suture control device is positioned so that it does not extend above the planar surface of the annular frame.

4. The surgical retractor of claim 1, wherein the at least one suture control device and the notch in which it is positioned are formed as a unitary construction.

5. The surgical retractor of claim 1, wherein the at least one suture control device is attached within at least one of the notches.

6. The surgical retractor of claim 1, wherein at least one of the notches in the annular frame does not include a suture control device.

7. A surgical retractor system comprising:
an annular frame having an inner edge, an outer edge having a plurality of spaced notches, and first and second opposite surfaces, wherein the inner edge defines an inner area of the frame; and
at least one suture control device extending from at least one of the first and second opposite surfaces for capturing at least one suture.

8. The surgical retractor system of claim 7 wherein the at least one suture control device comprises a raised knob portion.

9. The surgical retractor system of claim 8, wherein the raised knob portion is spaced from the opposite first or second surfaces of the annular frame by a space that is sufficient to retain a suture in the space.

10. The surgical retractor system of claim 8, wherein the raised knob portion comprises at least one notch.

11. The surgical retractor system of claim 10, wherein the at least one notch is substantially parallel to one of the first and second opposite surfaces of the annular frame.

12. The surgical retractor system of claim 10, wherein the at least one notch is substantially perpendicular to one of the first and second opposite surfaces of the annular frame.

13. The surgical retractor system of claim 8, wherein the raised knob portion comprises at least one channel.

14. The surgical retractor system of claim 7, wherein the at least one suture control device is made of silicone.

15. The surgical retractor system of claim 7, wherein the at least one suture control device is a coiled member.

16. The surgical retractor system of claim 15, wherein the coiled member comprises at least two adjacent solid members and a gap between the at least two adjacent solid members for capturing at least one suture.

17. A surgical retractor system comprising an annular frame having an inner edge, an outer edge having a plurality of spaced notches having a first width and at least one suture control notch having a second width that is less than the first width, wherein at least one suture control notch is spaced from a notch of the annular frame.
